(19) United States
(12) Patent Application Publication
(10) Pub. No.: US 2016/0007921 A1
(43) Pub. Date: Jan. 14, 2016

(54) HEAD-MOUNTED NEUROLOGICAL ASSESSMENT SYSTEM

(71) Applicant: Vivonics, Inc., Waltham, MA (US)

(72) Inventors: Anna M. Galea, Stow, MA (US); Michael Cevette, Scottsdale, AZ (US); Jamie Bogle, Scottsdale, AZ (US); Ken Brookler, Norwalk, CT (US)

(73) Assignee: Vivonics, Inc.

(21) Appl. No.: 14/795,044

(22) Filed: Jul. 9, 2015

Related U.S. Application Data
(60) Provisional application No. 62/023,021, filed on Jul. 10, 2014.

(51) Int. Cl. A61B 5/00; A61B 5/0484; A61B 3/02; A61B 3/113; A61B 3/032; A61B 5/03; A61B 5/0488

(52) U.S. Cl. CPC A61B 5/6814; A61B 5/0075; A61B 5/0082; A61B 5/031; A61B 5/04842; A61B 5/0488; A61B 5/4863; A61B 5/4023; A61B 3/113; A61B 3/032; A61B 3/02

(57) ABSTRACT

A head-mounted neurological assessment system including a head-mounted frame adapted to fit on a head of a user. One or more sensors are configured to measure parameters associated with an injured brain and/or vestibular system of the user. A display device is coupled to the frame and proximate eyes of the user. A processor subsystem is coupled to the one or more sensors and the display device and configured to perform tests for monitoring the function of an injured brain and/or vestibular system of the user.

### RELATED APPLICATIONS

This application hereby claims the benefit of and priority to U.S. Provisional Application Ser. No. 62/023,021, filed on Jul. 10, 2014 under 35 U.S.C. §§119, 120, 363, 365, and 37 C.F.R. §1.55 and §1.78 and incorporated herein by this reference.

### GOVERNMENT RIGHTS

This invention was made in part with U.S. Government support under Contract No. W81XWH-14-C-0009, awarded by the U.S. Army. The Government may have certain rights in certain aspects of the subject invention.

### FIELD OF THE INVENTION

This invention relates to a head-mounted neurological assessment system.

### BACKGROUND OF THE INVENTION

Brain injury is now recognized as the signature wound from modern warfare. According to the Defense and Veterans Brain Injury Center there were 266,810 brain injuries recorded in the U.S. Military between 2000 and 2012. The problem is not confined to warfare, as stateside personnel and military personnel are at a higher risk than the general population to experience traumatic brain injury (TBI). This may be due to a number of factors including the population demographics of the military (in general, young athletic males are a high-risk category for TBI) and the strenuous activities and duties of military service playing key roles. Active civilians, particularly those participating in competitive sports, are also at risk for TBI.

A closed-head brain injury, whether incurred as a result of blunt force trauma or a blast wave, can have insidious effects on the soldier or athlete. Even mild TBI (mTBI) can have considerable long-term sequelae. The correlation between mTBI and increased risk for Post-Traumatic Stress Disorder (PTSD) is known, although not fully understood. Although many casualties may suffer from headache or dizziness, it is difficult with conventional systems to image every soldier or athlete who experiences a potential brain injury. Most conventional imaging systems are large and require significant power. Moreover, damage to delicate brain tissues is frequently undetectable by conventional imaging, including CT scanning, and the like, even when such imaging is available.

In many active populations, especially the military and participants in professional or academically-sponsored competitive sports, the injured person may try to shrug off the seemingly mild symptoms of headache, dizziness, and the like. However, an unknown percentage of those injured have experienced clinically significant brain injury, which if left untreated, may worsen or at least make permanent some damage.

Some preferred conventional systems to identify which casualties are at the most risk of brain injury are those that monitor the physical trauma (such as blast waves or impact) that the head experiences. However such conventional systems may only provide information based on an empirical diagnostic technique which may not take into account individual variability with regards to susceptibility of brain injury. Thus, two people experiencing the same physical trauma are likely to exhibit different levels of damage. Without a direct measure of the damage, these individuals may be impossible to differentiate. One safe way to utilize such a conventional system is to treat each individual as though they were at the most delicate or vulnerable end of the scale. However, this cautious approach results in unnecessary therapy for a significant portion of the population.

Conventional post-injury cognitive tests, such as the Sport Concussion Assessment Tool 2 (SCAT-2) may be used to help triage casualties. However these conventional tests may have significant drawbacks, including a learning effect where an athlete will score better on the test with repeat exposure which may offset and mask the effect of concussive events, the tests take too long to administer, the tests require a baseline measure which is either not available or may adversely impact the test itself due to the learning effect, and damage to the deeper structures of the brain is not necessarily identifiable in a test of cognition.

Visual Evoked Potentials (VEP) and Intracranial Pressure (ICP) are two tests that may be used to indicate the presence of clinically significant brain injury. In a VEP test, the shape and latency of the electrical response at the occipital cortex from a visual stimulus is measured. This may provide a sensitive indication of visual pathway disturbances as they traverse through the parietal and temporal lobes to their final destination in the occipital lobes. Increased ICP, which has been shown to have a positive correlation to VEP latency, may also serve as a test for brain injury. Conventional tests for VEP and ICP are not practical for use in a far-forward military or an athletic sideline setting. VEP equipment generally uses a large computer monitor and sensitive recording equipment. Conventional systems and methods for measuring ICP are invasive because they require direct access to the brain by penetrating the skull. Although there have been attempts made at miniaturizing VEP equipment and implementing a non-invasive ICP recording system, none have yet materialized to the point of beginning the FDA process for eventual approval for distribution as a useful medical device.

In addition to cognitive deficits, mild traumatic brain injury (MTBI) frequently leaves subtle balance dysfunctions that are difficult to measure, assess, and treat. MTBI commonly leads to high rates of dizziness, imbalance, and vertigo

Traditional measures of peripheral vestibular function (i.e., caloric testing) are highly variable in this population.

Otolith function is similarly variable. Overall, peripheral vestibular dysfunction may be quite common following MTBI, but the clinical presentation is inconsistent.

In an active population, such as that of the U.S. Military or organized athletics, the dizziness and unsteadiness that often accompany MTBI may be devastating to the quality of life. Balance dysfunction is often associated with poor recovery prognosis and may be persistent for years following the initial injury. Abnormalities found during postural evaluation range from peripheral vestibular involvement to involvement of the entire balance system (visual, vestibular, somatosensory). This disconnection between sensory inputs requires the brain to choose which input is dominant. If the vestibular system provides faulty or unreliable input, the brain selects a preference for strong visual inputs, which leads to imbalance in conditions with visual field provocation.

Although mTBI and its vestibular sequelae are a common problem, conventional diagnostic systems and methods suitable for use in forward clinics, sidelines or primary/urgent care facilities remain primitive at best. Accurate, objective diagnostics remain relegated to more sophisticated sensors with highly trained and specialized staff.

### BRIEF SUMMARY OF THE INVENTION

This invention features a head-mounted neurological assessment system. The system comprises a head-mounted frame adapted to fit on a head of a user. One or more sensors are configured to measure parameters associated with an injured brain and/or vestibular system of the user. A display device is coupled to the frame and proximate eyes of the user. A processor subsystem is coupled to the one or more sensors and the display device configured to perform tests for monitoring the function of an injured brain and/or vestibular system of the user.

In one embodiment, the one or more sensors may include one or more of: a plurality of near infrared (NIR) sensors, a near infrared spectroscopy (NIRS) sensor, a plurality of electroencephalogram (EEG) sensors, and/or a plurality of electromyography (EMG) sensors. The system may include one or more of: an accelerometer coupled to the processor subsystem and configured to determine motion of the head of the user, at least one camera coupled to the processor subsystem and configured to monitor movement of eyes of the user, a toggle switch coupled to the processor subsystem and configured to receive user input, and/or a stimulation device coupled to the processor subsystem for stimulating a predetermined location on the head. One NIR sensor coupled to the frame proximate an artery receiving blood which emanates from the cranial cavity, another NIR sensor and may be coupled to the frame proximate an artery which does not receive blood emanating from the cranial cavity, and another NIR sensor is coupled to a distal artery of the user. One EEG sensor may be coupled to the frame proximate the occipital region of the head, another EEG sensor may be coupled to the frame proximate a forehead of the user, and another EEG sensor may be coupled to the frame proximate side of the head. The NIRS sensor may be coupled to the frame proximate the occipital region of the head. The one or more tests may include: a test to determine intracranial pressure (ICP), a visually envoked potential (VEP) test, a visually envoked activation (VEA) test, a vestibular ocular reflex (VOR) test, a dynamic visual acuity test (DVAT), a Nystagmus test, a head thrust test, an oculometric evaluation test, a subjective visual vertical (SVV) test, a subjective visual horizontal (SVH) test, an otolith evaluation test, and a moving visual field test. The processor subsystem may be configured to monitor pulsations of an artery receiving blood which emanates from the cranial cavity and, an artery which does not receive blood emanating from the cranial cavity, and the distal artery to perform a test to determine ICP. The display device may be configured to display and flash one or more images to the user and the processor subsystem may be configured to perform a visually envoked potential (VEP) test in response to signals from EEG sensors. The display device may be configured to flash one or more images to the user and the processor subsystem may be configured to perform a visually envoked activation (VEA) test in response to signals from NIRS sensors. The processor subsystem may be responsive to signals from the accelerometer and at least one more camera may be configured to perform a vestibular ocular reflex (VOR) test. The processor subsystem may be responsive to signals from the accelerometer and the at least one camera and may be configured to perform a Nystagmus test and/or a head thrust test. The display device may be configured to display a non-vertical or non-horizontal straight line to the user and the toggle switch may be responsive to user input to adjust the location of non-vertical line or the non-horizontal line such that non-vertical line or the non-horizontal line appears vertical or horizontal to the user, and the processing subsystem may be configured to perform a subjective visual vertical (SVV) test and/or a subjective visual horizontal (SVH) test in response signals from the display device. The display device may be configured to display moving target for the user to follow and the processing subsystem may be configured to perform an oculometric evaluation test and/or an ocular counter roll test in response signals from the at least one camera. The display device may be configured to display an eye chart and the user reads the eye chart stationary and in motion and the processing subsystem may be configured to perform a dynamic visual acuity test (DVAT) in response signals from the at least one camera and the accelerometer. The stimulation device may provide a stimulus to a predetermined location on the head and the processing subsystem may measure a vestibular response from signals from the at least one camera or the EMG sensors to perform an otolith evaluation test. The display device may be configured to display moving target for the user to follow and the processing subsystem may be configured to perform a moving visual field test in response signals from the at least one camera and the accelerometer. The system may include an additional display device coupled to the processor system configured to output and display the tests for monitoring the function of the brain and/or vestibular system.

This invention also features a head-mounted neurological assessment system. The system comprises a head-mounted frame adapted to fit on a head of a user. A plurality of sensors including near infrared (NIR) sensors, electroencephalogram (EEG) sensors, and/or electromyography (EMG) sensors is configured to measure parameters associated with an injured brain and/or vestibular system of the user. A display device is coupled to the frame and proximate eyes of the user, and a processor subsystem is coupled to the plurality of sensors and the display device configured to perform tests for monitoring the function of an injured brain and/or vestibular system of the user.

This invention also features a head-mounted neurological assessment system. The system comprises a head-mounted frame adapted to fit on a head of a user. A plurality of sensors configured to measure parameters associated with an injured brain and/or vestibular system of the user. One or more sensors is configured to measure parameters associated with an injured brain and/or vestibular system of the user. A display device is coupled to the frame and proximate eyes of the user. An accelerometer is coupled to the processor subsystem configured to determine motion of the head of the user. At least one camera is coupled to the processor subsystem configured to monitor movement of eyes of the user, and a processor subsystem is coupled to the one or more sensors and the display device and configured to perform tests for monitoring the function of an injured brain and/or vestibular system of the user.

### BRIEF DESCRIPTION OF THE SEVERAL VIEWS OF THE DRAWINGS

Other objects, features and advantages will occur to those skilled in the art from the following description of the embodiments and the accompanying drawings, in which:

FIG. 1A is a three-dimensional front-side view showing the primary components of one embodiment of the head-mounted neurological assessment system of this invention;

FIG. 1B is a three-dimensional side-view of the system shown in FIG. 1A;

FIG. 1C is a three-dimensional back-view of the system shown in FIG. 1A;

FIG. 2 is a schematic block diagram showing one embodiment of the system shown in FIGS. 1A-1C;

FIG. 3 shows examples of EEG signals exposed to a flash by the display device shown in one or more of FIGS. 1A-2 and an example of a visually envoked potential (VEP) diagram;

FIG. 4 depicts examples of a head motion waveform and eye motion waveform used by the system shown in one or more of FIGS. 1A-2 to perform a vestibular ocular reflex (VOR) test;

FIG. 5 shows an example of an eye motion waveform used by the system shown in one or more of FIGS. 1A-2 to perform a Nystagmus test;

FIG. 6 shows an example of an eye chart that may be displayed by the display device shown in one or more of FIGS. 1A-6 to conduct a dynamic visual acuity test (DVAT).

FIG. 7 shows an example of a moving target that may be used by the system shown in one or more of FIGS. 1A-2 to perform an ocular metric evaluation test;

FIG. 8 depicts examples of non-vertical and non-horizontal images used by the system shown in one or more of FIGS. 1A-2 to perform a subjective visual vertical (SVV) test and/or a subjective visual horizontal (SVH) test; and

FIG. 9 shows an example of a moved image that may be used by the system in one or more of FIGS. 1A-2 to perform a moving visual field test.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

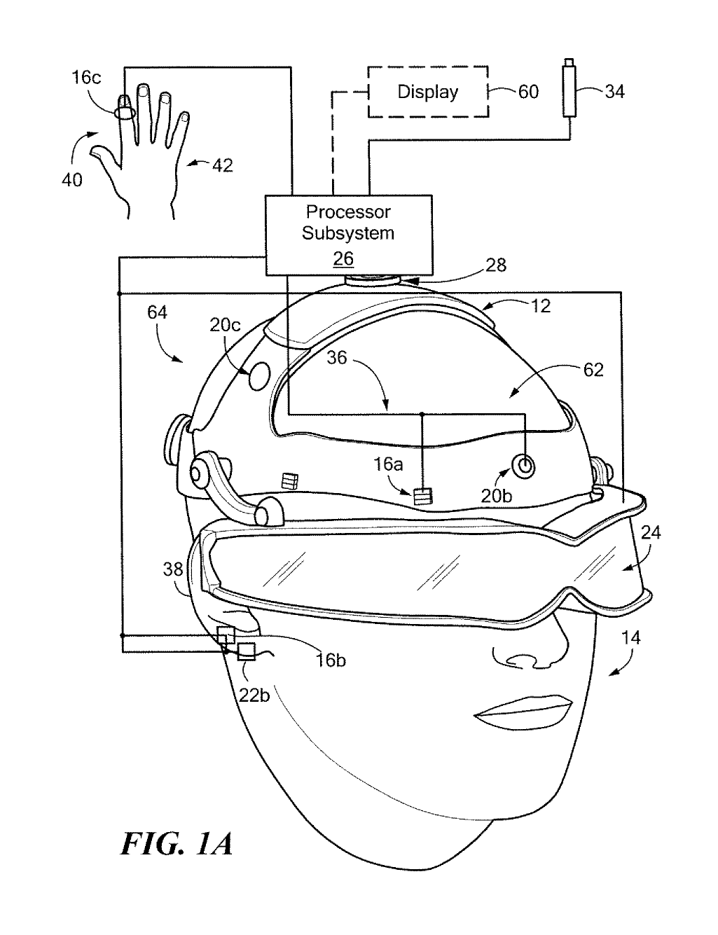

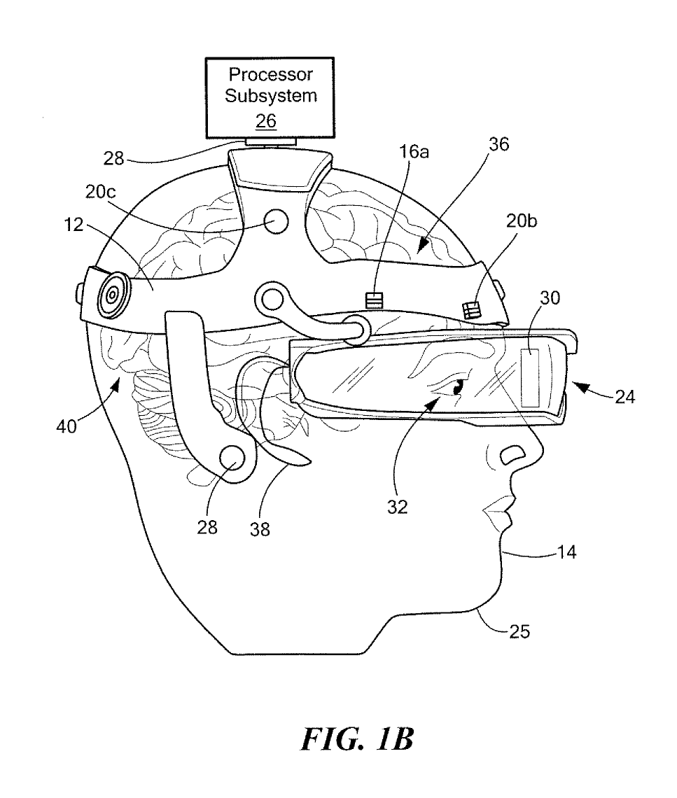

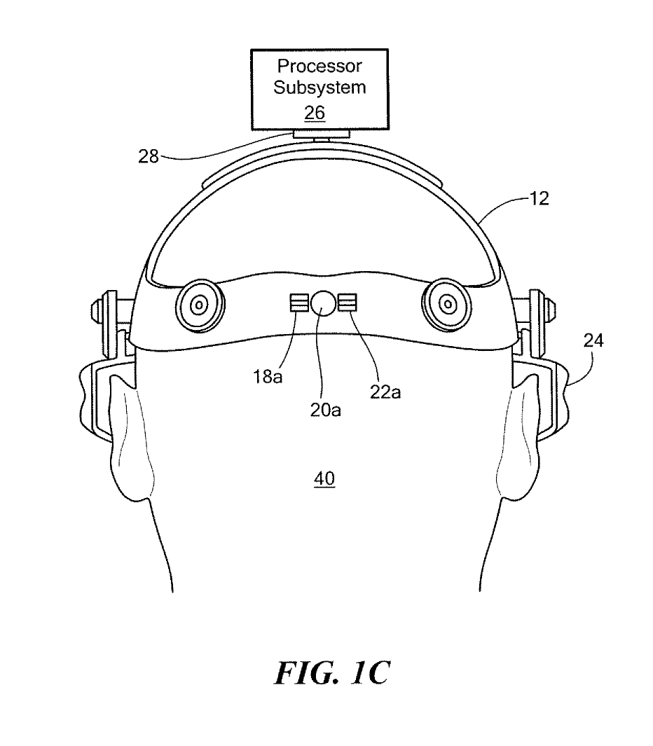

FIGS. 1A-1C show one embodiment of head-mounted neurological assessment system 10 of this invention. System 10 includes head-mounted frame 12 adapted to fit on head 14 of the user as shown. System 10 also includes one or more sensors configured to measure parameters associated with an injured brain and/or vestibular system of the user. In one example, the sensors may include near infrared (NIR) sensors 16a, 16b, 16c, near infrared spectroscopy (NIRs) sensor 18, a plurality of electroencephalogram (EEG) sensors 20a, 20b, and 20e, and a plurality of electromyography (EMG) sensors 22a, 22b, discussed in further detail below. System 10 further includes display device 24 coupled to frame 12 and proximate the eyes of a user, e.g., the eyes exemplarily indicated at 32, FIG. 1B, as shown. System 10 also includes processor subsystem 26 coupled to one or more of sensors 16a, 16b, 16c, 18, 20a, 20b, 20c and/or 22a, 22b and display device 24. Processor subsystem 26, preferably includes one or more processors, a computing device, an application specific integrated circuit (ASIC) or similar type device, firmware, hardware, and/or software (including firmware, resident software, microcode, and the like) which execute instructions to perform one or more tests for monitoring the function of an injured brain and/or vestibular system of head 14 of the user.

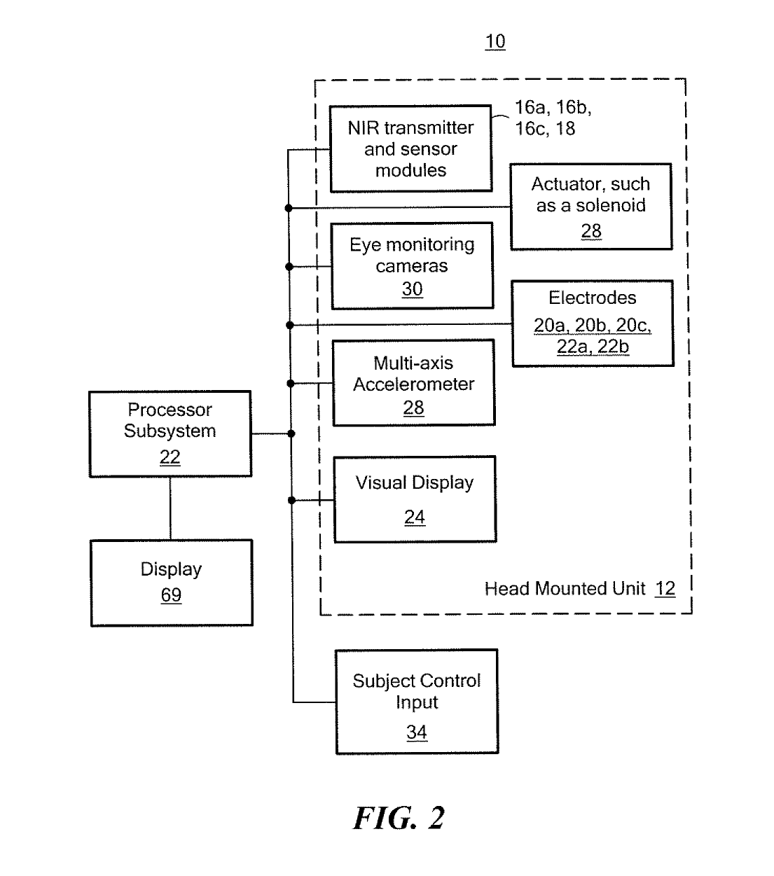

Head-mounted neurological assessment system 10 may also include accelerometer 28, preferably coupled to frame 12 as shown, which measures the motion of head 14 of the user. System 10 may also include at least one camera 30, FIG. 1B, preferably coupled within display 24 as shown, which monitors the movement of eyes 32 of the user. Preferably, camera 30 is an infrared eye-monitoring camera and preferably includes video-Nystagmography technology. System 10 may include two cameras 30, which each monitor movement of one of the eyes of the user. System 10 may also include toggle switch 34, FIG. 1A, coupled to processor subsystem 26 as shown which receive input from hand 42 of the user. System 10 may also include stimulation device 28, FIG. 1B, e.g., a solenoid or similar stimulation device, preferably coupled to frame 12 as shown, configured to stimulate a predetermine location on the head, e.g., in this example, behind ear 34 of head 14 of the user as shown. System 10 may further include display 60 which can output and display the results of the tests performed by processor subsystem 26. FIG. 2, where like parts include like numbers, is a schematic block diagram showing one example of the primary components of system 10 shown in FIGS. 1A-1C.

In one example, system 10, FIGS. 1A-1C, may include EEG sensor 20a (active) coupled to frame 12 proximate occipital region 40 (shown more clear in FIG. 1B), EEG sensor 20b (reference), FIG. 1A, coupled to frame 12 proximate forehead 62 of the user and EEG sensor 20c (ground) coupled to frame 12 proximate side 64 of head 14. System 10 may also include near infrared spectroscopy (NIRS) sensor 18, FIG. 1C, preferably coupled to frame 12 proximate occipital region 40, FIGS. 1B-1C.

The tests for monitoring the function of an injured brain and/or vestibular system performed by system 10 may include a test to determine ICP, a visually evoked potential (VEP) test, a vestibular ocular reflex (VOR) test, a dynamic visual acuity test (DVAT) test, and Nystagmus test, a head thrust test, an ocular metric evaluation test, a subjective visual vertical field (SVV) test, a subjective visual horizontal (SVH) test, an otolith evaluation test, and a moving visual field test.

For example, to perform a test for ICP, NIR sensor 16a may be coupled to frame 12 proximate an artery receiving blood which emanates from the cranial cavity, e.g., the supraorbital artery, such as on temple 36, FIG. 1A, of the user as shown, NW sensor 16b may be placed proximate an artery which does not receive blood emanating from the cranial cavity, e.g., the external carotid artery, e.g., near ear 38 of the user, and sensor 16c may be placed on a distal artery, e.g., on finger 40 of hand 42 of the user. In this example, processor subsystem 26 receives signals from NW sensors 16a, 16, and 16e and

is configured to monitor pulsations of the artery receiving blood which emanates from the cranial cavity and, the artery which does not receive blood emanating from the cranial cavity, and the distal artery performs a test to determine ICP. Additional details of the test for determining ICP from NIR sensors 16, a, 16b, and 16c is disclosed in applicant's co-pending application Publ. No. 2015-0018697, incorporated by reference herein.

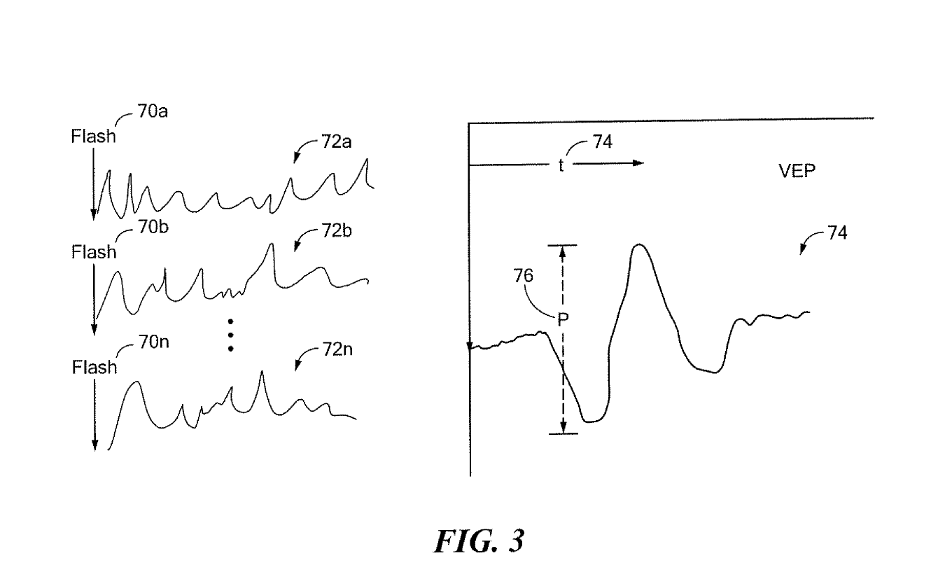

System 10 may use display device 24, FIGS. 1A-1C, to flash one or more images at the eyes of the user. For this test, processor subsystem 26 is configured to perform a visually envoked potential (VEP) test in response to signals from EEG sensors 20a, 20b, and 20c. For example, display device 24 may flash an image at the eyes of user indicated at 70a, FIG. 3, which affects EEG signal 72a. Another image is flashed to the user, indicated at 70b, which affects EEG signal 72b. The process is repeated numerous times, e.g., about 40-500, times indicated by flashes 70n and EEG image 72n. The result is VEP graph 74 which includes measurements for time, indicated at 74, and potential, indicated at 76 which may be used by system 10 to test the VEP of the user.

Similarly, system 10 may also use display device 24, FIGS. 1A-IC, to flash one or more images at the eyes of the user and processor subsystem 26 performs a visually evoked activation (VEA) test in response to signals from NIRS sensor 18, FIG. 1C. NIRS sensor 18 preferably records the increase in the cortical supply of oxygenated blood and may provide latency information similar to that of the EEG-recorded VEP. However, the baseline for the latency is expected to be greater.

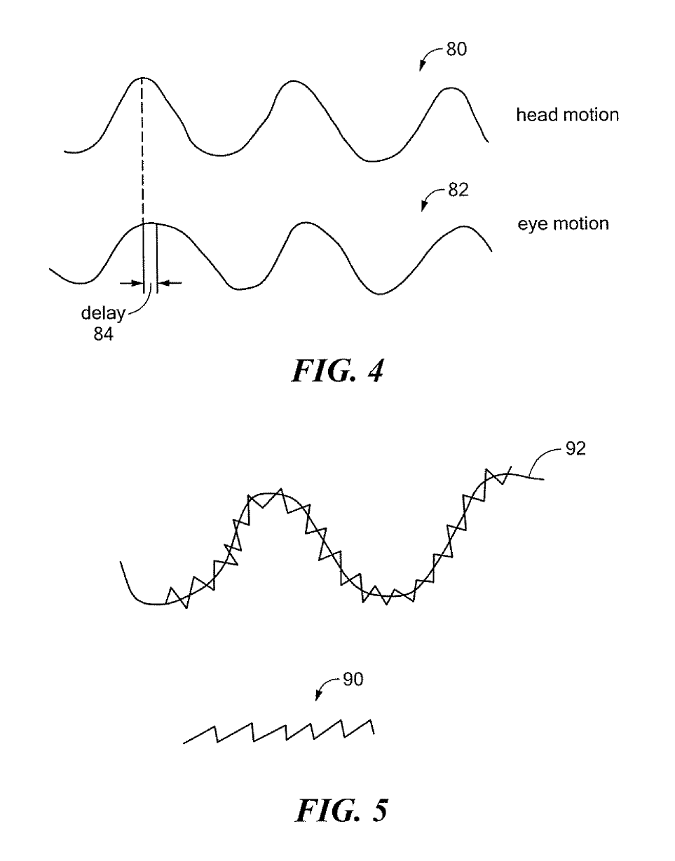

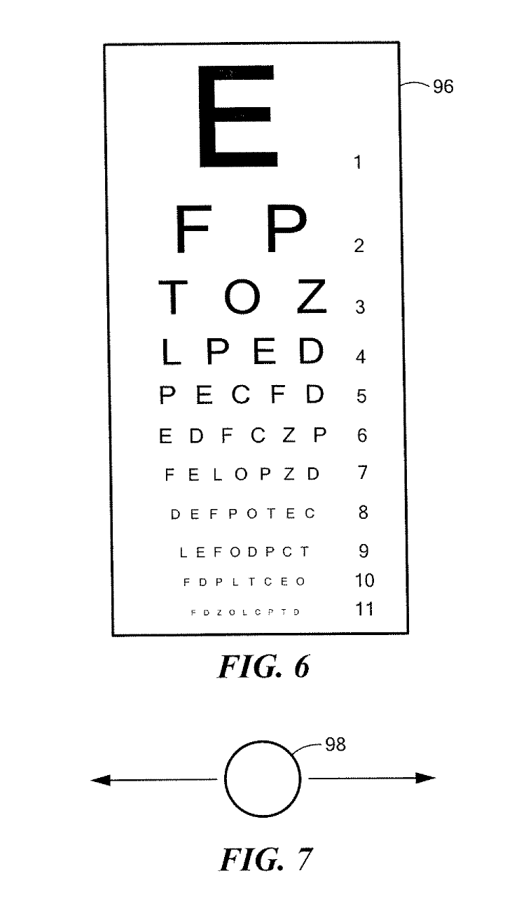

To perform a vestibular ocular reflex (VOR) test, system 10 measures the motion of head 14 using accelerometer 28 and the motion of the eyes using at least one camera 30 when the user is presented a moving visual target and is instructed to follow the target without moving the head. Processor subsystem 26 is responsive to signals from accelerometer 28 and at least one camera 30 and performs the VOR test. The monitoring of eye movements with currently available video-Nystagmography technology which may be incorporated into at least one camera 30 allows for evaluation VOR. Healthy people have a clear coupling between the head motion signal 80, FIG. 4, and eye motion signal 82 and have small delay 84 or no coupling. In unhealthy individuals, delay 84 is much bigger. The vestibular ocular reflex serves to maintain fixation on a visual target despite active or passive head movement and serves to maintain the orientation of the horizontal meridian of the retina with the horizon. Disruption of this reflex can provoke post traumatic dizziness and significant loss of balance. Common signs of disruption include either the loss of VOR based compensatory eye movements with head movement or the inclusion of aberrant VOR based eye movements (Nystagmus) when the head not moving. The Nystagmus and/or a head thrust test discussed below can be integrated for use in the evaluation of VOR behavior in acute casualty patients. Video-Nystagmography based methods which may be included using one camera 30 which have been successfully used in modern clinical settings, housed in a unit capable of being deployed in forward medical settings.

Nystagmus is small saccadic motions of the eye in response to motion of the head that often develops when the VOR is acutely impaired. In injured people, Nystagmus may be present even when they are standing still. To perform a Nystagmus test, system 10 monitors the motion of head 14 using accelerometer 28 and the motion of eyes 32 using at least one camera 30 when the user is stationary. Nystagmus are small tooth like patters, e.g., as indicated at 90, FIG. 5, that can be superimposed on any slower eye motion signal 90 as shown. Processor subsystem 26 is responsive to signals from accelerometer 28 and at least one camera 30 and performs the Nystagmus test. The Nystagmus test may also be conducted after motion as been induced on the user, e.g. by rotating the user in a chair. The Nystagmus test will inventory (detect, record and quantify) the presence of aberrant Nystagmus that may be seen without provocation (i.e., spontaneously with and without gaze fixation), or provoked by changing head position (i.e., position and positioning provoked). Analysis will focus on identifying abnormal Nystagmus that may indicate acute labyrinthine trauma.

Similarly, system 10 may use accelerometer 28 and at least one camera 30 to perform a head thrust test. The head thrust test may is used to detect semicircular canal dysfunction. In this test, the patient is asked to focus on a stationary point and the head is then moved. A trained clinician watches for saccadic motions of the eyes that account for the head movement while accelerometer 28 and at least one camera 30 monitor the motion of head 14 and eyes 32 and processor subsystem 26 performs the head thrust test.

Additionally, display device 24 may be configured to display eye chart 96, FIG. 7 and the user is instructed to reads eye chart 96 while stationary and in motion. Processing subsystem 26 performs a dynamic visual acuity test (DVAT) in response to the signals from the at least one camera 30 and accelerometer 28.

System 10 may also perform ocular metric evaluation test. To do this, display device 24 displays a moving target, e.g., moving target 98, FIG. 7, to the user and the user is asked to follow the target with his or her eyes to the best of his or her ability while keeping the head stationary. At least one camera 30 monitors the motion of the eye movement of the user. Processor subsystem 28 is responsive to signals from the at least one camera 30 and performs the ocular metric evaluation test. This ocular metric evaluation test allows for evaluation of oculometric function, such as vertical and horizontal smooth pursuit, tracking, and optokinetic Nystagmus systems. In a similarly manner, system 10 may perform an ocular counter roll test. Ocular counter roll is an otolithic reflex generated to maintain posture and gaze. To conduct this test, the user is asked to tilt the head laterally in order to observe the expected ocular counter roll. At least one camera 30 monitors the movement of the eyes and processor subsystem 26 performs the ocular counter roll test.

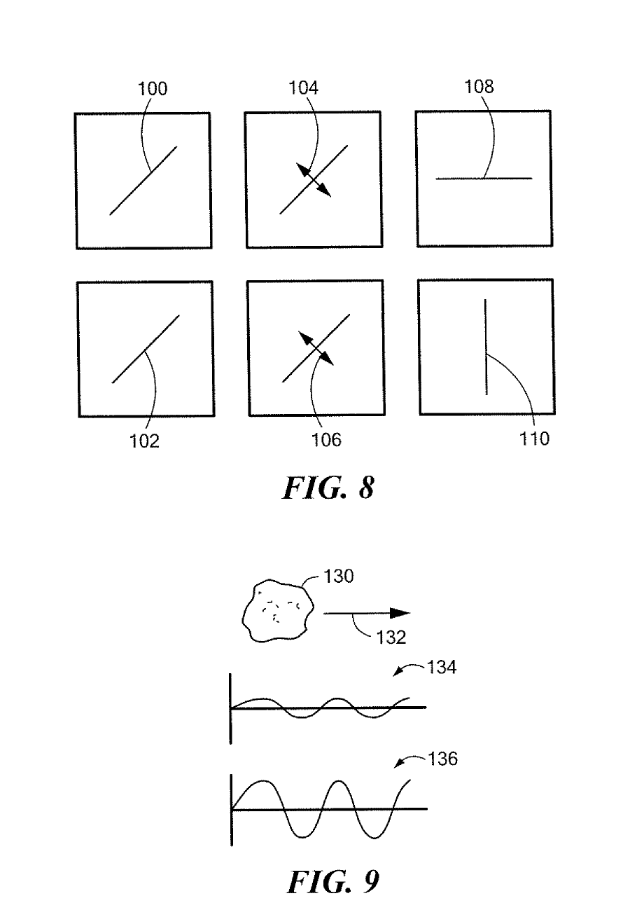

Acute vestibular dysfunction may cause several compelling illusions that correlate with the severity of acute labyrinthine trauma. To test for vestibular dysfunction, system 10 can perform a subjective visual vertical (SVV) test and/or a subjective visual horizontal (SVH). To perform the SVV and/or SVH test, display device 24, FIGS. 1A-1C, displays a non-vertical or non-horizontal straight line to the user, e.g., non-vertical or non-straight line 100, 102, FIG. 8, to the user. The user then uses toggle switch 34, FIG. 1A, to adjust the location of non-vertical line 100 or non-horizontal line 102, indicated at 106, 106, respectively, such that the non-vertical or non-horizontal line appears to be vertical or horizontal to the user, as indicated at 108, 110, respectively. Processing subsystem 26 is responsive to the adjusted non-vertical line 108 or adjusted non-horizontal line 110 provided by display device 24 and performs the SVV test and/or the SVH.

System 10 may also perform an otolith evaluation test. The otolith system within the vestibular labyrinth controls muscle tone and balance when standing, walking or running. Acute disorders of this system can result in dizziness, decreased situational awareness and falls. To perform an otolith evaluation test, system 10 uses stimulation device 28, FIG. 1B, preferably located behind ear 38 as shown to generate a myogenic response from the vestibular system of the user. Processor subsystem 26 measures the vestibular response from signals from the at least one camera 30 or EMG sensors 22a, 22b to perform the otolith evaluation test. In healthy people, there is both Nystagmus response and an EMG response. In injured people, there is an aberrant or absent response. Thus, system 10 can assess vestibular otolith-spinal reflex function by incorporating measurement of the cervical vestibular evoked myogenic potential (cVEMP) and the ocular vestibular evoked myogenic potential (oVEMP) using EMG sensors 22a, 22b.

In yet another example, system 10 may perform a moving visual field test. To do this, display device 24 displays an image, e.g. image 130, FIG. 9, to the eyes of the user. Image 130 is then moved, e.g., as indicated by arrow 132, and the user is instructed to follow image 130. The balance of the user is then monitored with accelerometer 28, FIGS. 1A-1C, to see if the subject has lost his or her balance or falls down. Processing subsystem 26 performs a moving visual field test in response to signals from accelerometer 28. FIG. 9 shows an example of accelerometer signal 134 for a health person and accelerometer signal 136 for an injured person. The balance response indicates the presence or absence of vestibular dysfunction.

System 10, FIG. 1, may also include an additional display device 100, FIGS. 1A and 2, that can output and display all of the tests discussed above for monitoring the function of the brain and/or vestibular system.

The result is head-mounted neurological assessment system 10 has combined for the first time head-mounted frame 12, near infrared (NIR) sensors 16a, 16b, 16c, near infrared spectroscopy (NIRS) sensor 18, electroencephalogram (EEG) sensors 20a, 20b, and 20c, and/or electromyography (EMG) sensors 22a, 22b which effectively and efficiently measure parameters associated with an injured brain and/or vestibular system of the user, a display device, a processor subsystem and preferably an accelerometer, camera, stimulation device. System 10 can perform tests which efficiently and effectively monitor the many facets of the injured brain function and/or vestibular system. System 10 is simple to use, fast, accurate and requires minimal training, and can be performed in military settings and athletic sideline settings without the need for large and expensive imaging systems, and computer monitoring and recording equipment. System 10 also provides repeatable quantitative test that can provide information as to a function of an injured brain and/or vestibular system.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

Other embodiments will occur to those skilled in the art and are within the following claims.

What is claimed is:

1. A head-mounted neurological assessment system comprising:
a head-mounted frame adapted to fit on a head of a user;
one or more sensors configured to measure parameters associated with an injured brain and/or vestibular system of the user;
a display device coupled to the frame and proximate eyes of the user; and
a processor subsystem coupled to the one or more sensors and the display device configured to perform one or more tests for monitoring the function of an injured brain and/or vestibular system of the user.

2. The system of claim 1 in which the one or more sensors include one or more of: a plurality of near infrared (NIR) sensors, a near infrared spectroscopy (NIRS) sensor, a plurality of electroencephalogram (EEG) sensors, and/or a plurality of electromyography (EMG) sensors.

3. The system of claim 1 further including one or more of: an accelerometer coupled to the processor subsystem configured to determine motion of the head of the user, at least one camera coupled to the processor subsystem configured to monitor movement of eyes of the user, a toggle switch coupled to the processor subsystem configured to receive user input, and/or a stimulation device coupled to the processor subsystem for stimulating a predetermined location on the head.

4. The system of claim 2 in which one NIR sensor is coupled to the frame proximate an artery receiving blood which emanates from the cranial cavity, another NIR sensor and is coupled to the frame proximate an artery which does not receive blood emanating from the cranial cavity, and another NIR sensor is coupled to a distal artery of the user.

5. The system of claim 2 in which one EEG sensor is coupled to the frame proximate the occipital region of the head, another EEG sensor and is coupled to the frame proximate a forehead of the user; and another EEG sensor is coupled to the frame proximate side of the head.

6. The system of claim 2 in which NIRS sensor is coupled to the frame proximate the occipital region of the head.

7. The system of claim 1 in which the one or more tests include: a test to determine intracranial pressure (ICP), a visually envoked potential (VEP) test, a visually envoked activation (YEA) test, a vestibular ocular reflex (VOR) test, a dynamic visual acuity test (DVAT), a Nystagmus test, a head thrust test, an oculometric evaluation test, a subjective visual vertical (SVV) test, a subjective visual horizontal (SVH) test, an otolith evaluation test, and a moving visual field test.

8. The system of claim 4 in which the processor subsystem is configured to monitor pulsations of an artery receiving blood which emanates from the cranial cavity and, an artery which does not receive blood emanating from the cranial cavity, and the distal artery to perform a test to determine ICP.

9. The system of claim 5 in which the display device is configured to display and flash one or more images to the user and the processor subsystem is configured to perform a visually envoked potential (VEP) test in response to signals from EEG sensors

10. The system of claim 2 in which the display device is configured to flash one or more images to the user and the processor subsystem is configured to perform a visually envoked activation (YEA) test in response to signals from NIRS sensors

11. The system of claim 3 in which the processor subsystem is responsive to signals from the accelerometer and at least one more camera is configured to perform a vestibular ocular reflex (VOR) test.

12. The system of claim 3 in which the processor subsystem is responsive to signals from the accelerometer and the at least one camera and is configured to perform a Nystagmus test and/or a head thrust test.

13. The system of claim 3 in which the display device is configured to display a non-vertical or non-horizontal straight line to the user and the toggle switch is responsive to user input to adjust the location of non-vertical line or the non-horizontal line such that non-vertical line or the non-horizontal line appears vertical or horizontal to the user, the processing subsystem configured to perform a subjective visual vertical (SVV) test and/or a subjective visual horizontal (SVH) test in response signals from the display device.

14. The system of claim 3 in which the display device is configured to display moving target for the user to follow and the processing subsystem configured to perform a an oculometric evaluation test and/or an ocular counter roll test in response signals from the at least one camera.

15. The system of claim 3 in which the display device is configured to display an eye chart and the user reads the eye chart stationary and in motion and the processing subsystem configured to perform a dynamic visual acuity test (DVAT) in response signals from the at least one camera and the accelerometer.

16. The system of claim 3 in which the stimulation device provides a stimulus to a predetermined location on the head and the processing subsystem measures a vestibular response from signals from the at least one camera or the EMG sensors to perform an otolith evaluation test.

17. The system of claim 3 in which the display device is configured to display moving target for the user to follow and the processing subsystem configured to perform a moving visual field test in response signals from the at least one camera and the accelerometer.

18. The system of claim 1 further including an additional display device coupled to the processor system configured to output and display the one or more tests for monitoring the function of the brain and/or vestibular system.

19. A head-mounted neurological assessment system comprising:
a head-mounted frame adapted to fit on a head of a user;
a plurality of sensors including near infrared (NIR) sensors, electroencephalogram (EEG) sensors, and/or electromyography (EMG) sensors configured to measure parameters associated with an injured brain and/or vestibular system of the user;
a display device coupled to the frame and proximate eyes of the user; and
a processor subsystem coupled to the plurality of sensors and the display device configured to perform tests for monitoring the function of an injured brain and/or vestibular system of the user.

20. A head-mounted neurological assessment system comprising:
a head-mounted frame adapted to fit on a head of a user;
a plurality of sensors configured to measure parameters associated with an injured brain and/or vestibular system of the user;
one or more sensors configured to measure parameters associated with an injured brain and/or vestibular system of the user;
a display device coupled to the frame and proximate eyes of the user;
an accelerometer coupled to the processor subsystem configured to determine motion of the head of the user,
at least one camera coupled to the processor subsystem configured to monitor movement of eyes of the user, and
a processor subsystem coupled to the one or more sensors and the display device configured to perform tests for monitoring the function of an injured brain and/or vestibular system of the user.
